(19) United States
(12) Patent Application Publication
(10) Pub. No.: US 2014/0202468 A1
(43) Pub. Date: Jul. 24, 2014

(54) FLEXIBLE UNDERGARMENT FOR PREVENTING THE SPREAD OF SEXUALLY TRANSMITTED DISEASES DURING SEXUAL ACTIVITY

(71) Applicant: T. John Parsi, San Antonio, TX (US)

(72) Inventor: T. John Parsi, San Antonio, TX (US)

(21) Appl. No.: 13/746,028

(22) Filed: Jan. 21, 2013

(51) Int. Cl. A61F 6/04; A61F 6/06

(52) U.S. Cl. CPC A61F 6/04; A61F 6/06
USPC 128/845; 427/2.3

(57) ABSTRACT

A flexible undergarment that is worn prior to a date under a normal undergarment. The flexible undergarment has a front portion connected with a back portion, forming a hip portion and two leg portions. The flexible undergarment covers at least a portion of the wearer's lower body. The flexible undergarment includes an integral front extension or an integral middle extension. The flexible undergarment has an integral back extension. The flexible undergarment provides protection from diseases associated with contact of bodily fluid, other people's skin, or the like.

### FIELD

The present embodiments generally relate to a flexible undergarment that can prevent the spread of sexually transmitted diseases during sexual activity and the method for making the flexible undergarment.

### BACKGROUND

A need exists for a flexible undergarment that prevents the transmission of sexually transmitted diseases due to bodily fluid contacting the skin and skin-to-skin contact while providing tactile sensations that do not limit sexual performance.

A need exists for a flexible undergarment that is a unitary tight fitting garment that fits snugly over the body with the same level of protection on all parts.

A need exists for a flexible undergarment that is an elastic garment made of material that enables the wearer to provide tactile sensations as if the wearer was not wearing any garment at all. The elastic garment fits like a glove, akin to biker shorts, but without being of a thick material.

A need exists for a flexible undergarment that is disposable after use and captures body fluid.

The present embodiments meet these needs.

### BRIEF DESCRIPTION OF THE DRAWINGS

The detailed description will be better understood in conjunction with the accompanying drawings as follows:

FIG. 1A depicts a front view of a flexible male undergarment on a male.

FIG. 1B depicts a side view of view of the flexible male undergarment on a male.

FIG. 2 depicts a side view of the flexible male undergarment.

FIG. 3A depicts a front view of a flexible female undergarment on a female.

FIG. 3B depicts a side view of the flexible female undergarment on a female.

FIG. 4 depicts a side view of the flexible female undergarment.

The present embodiments are detailed below with reference to the listed Figures.

### DETAILED DESCRIPTION OF THE EMBODIMENTS

Before explaining the present apparatus in detail, it is to be understood that the apparatus is not limited to the particular embodiments and that it can be practiced or carried out in various ways.

The present embodiments generally relate to a flexible undergarment that can prevent the spread of sexually transmitted diseases during sexual activity and the method for making the flexible undergarment.

The flexible undergarment can prevent transmission of sexually transmitted diseases (STD) through bodily fluid such as AIDS and syphilis and also prevent sexual infectious diseases like Human Papilloma Virus (HPV) that is transmitted through skin-to-skin contact. Protection from HPV is important because HPV can cause cervical cancer, oral cancer, and anal cancer.

In one or more embodiments, the flexible undergarment can cover a lower portion of a wearer's stomach, an upper portion of a wearer's thighs, the genitals and the anus of the wearer.

The flexible undergarment can be inexpensive to manufacture. The flexible undergarment can be made of any non-permeable material. For example, the flexible undergarment can be made of polymeric elastic compound and fabric that prevents the transmission of viruses, bacteria, funguses, other microbes, or combinations thereof.

In one or more embodiments of the flexible undergarment can be configured for use by a male.

In this embodiment, the flexible undergarment can be a one piece one layer flexible undergarment. The flexible undergarment can have a front portion and a back portion connected to the front portion, forming two leg portions and a hip portion configured to receive a lower portion of a body and to cover at least a portion thereof.

The male version of the flexible undergarment can have an integral front extension. The integral front extension can be formed at a location below the waist of a wearer and between the thighs of the wearer on the front side of the flexible undergarment. The integral front extension can be configured to receive a male organ of the wearer. The integral front extension can be formed during molding of the flexible undergarment.

The male version of the flexible undergarment can also include an integral back extension. The integral back extension can be formed at a location below the waist of and between the thighs of the wearer. The flexible undergarment can be located on the back side of the flexible undergarment. The integral back extension can be configured to receive a body part not of the wearer. The integral back extension can be formed during molding of the flexible undergarment. The integral back extension is configured to allow penetration of the wearer's anus and to prevent direct contact with the skin of the anus.

In one or more embodiments, the flexible undergarment can be configured for use by a female. In this embodiment, the flexible undergarment can have a front portion and a back portion connected to the front portion, forming two leg portions and a hip portion configured to receive a lower portion of a body and to cover at least a portion thereof.

The female version of the flexible undergarment can include an integral middle extension. The integral middle extension can be formed at a location below the waist of a wearer and between the thighs of the wearer. The integral middle extension can be configured to receive a body part not of the wearer. The integral middle extension can be formed during molding of the flexible undergarment. The integral middle extension can be configured to allow vaginal penetration of the wearer and to prevent direct contact with the skin of the vagina.

The female version of the flexible undergarment can also include an integral back extension. The integral back extension can be formed at a location below the waist of the wearer and between the thighs of the wearer. The integral back extension can be configured to receive a body part not of the wearer. The integral back extension can be formed during molding of the flexible undergarment. The integral back extension is configured to allow penetration of the wearer's anus and to prevent direct contact with the skin of the anus.

The flexible undergarment, in one or more embodiments, can be made from any material. Illustrative materials can include a polymeric material for preventing transmission of a virus, bacteria, fungus, or combinations thereof.

The polymeric material can have a thickness from about 1/64^{th} of an inch to about ¼ of an inch. Illustrative polymeric materials can include latex, polyurethane, polyisoprene, nitrile, polypropylene, polyamide, polyester, homopolymer, copolymers, and combinations thereof. The polymeric material can be a film.

In one or more embodiments, the polymeric material can be a natural rubber latex or a synthetic rubber latex or synthetic rubber. The synthetic rubber latex can be acrylonitrile, polyurethane, polychloroprene, or polyvinyl chloride.

The flexible undergarment can fit snugly against and partially cover a body of a wearer. The flexible undergarment can also allow for a tactile sensation to be provided to the wearer.

The flexible undergarment can cover a portion of the lower portion of the stomach, upper portion of the thighs, all of the genitals, and the anus.

The flexible undergarment can be seamless. In one or more embodiments, the flexible undergarment can have a thong shape, a brief shape, or a boxer shape.

The flexible undergarment can be worn underneath ordinary underwear prior to a date that might result in a sexual encounter.

The flexible undergarment can be manufactured using one or more methods.

One or more embodiments of the method can include pouring polymeric material into a mold, wherein the mold can be configured to form a flexible undergarment that has a front portion; a back portion connected to the front portion, forming two leg portions and a hip portion that can be configured to receive a lower portion of a body and to cover at least a portion thereof; an integral back extension on the back portion, wherein the integral back portion is configured to allow penetration of the wearer's anus and prevent direct contact with the skin of the anus; and an integral middle extension on the front portion, wherein the integral middle extension is configured to allow vaginal penetration of the wearer and prevent direct contact with the skin of the vagina, or an integral front extension on the front portion, wherein the integral front extension is configured to receive at least a penis of the wearer.

The method can also include drying the polymeric material forming the flexible undergarment.

The method can also include applying a moisturizing solution to the formed flexible undergarment. The moisturizing solution can be a powder like starch and calcium carbonate, or moisturizing solution like Aloe Vera, Hydrogel, Lanolin oil and vitamin E, or a solution having Aloe Vera, Lanolin oil, Vitamin E, or combinations thereof. The moisturizing solution can be applied to the formed flexible undergarment so that when the flexible undergarment is worn the moisturizing solution is in contact with a wearer's skin.

The method can also include drying the moisturizing solution.

An outer portion of the undergarment can have a flavor coating located thereon. The flavor coating can be sweet bubble gum, honey, or fruit products.

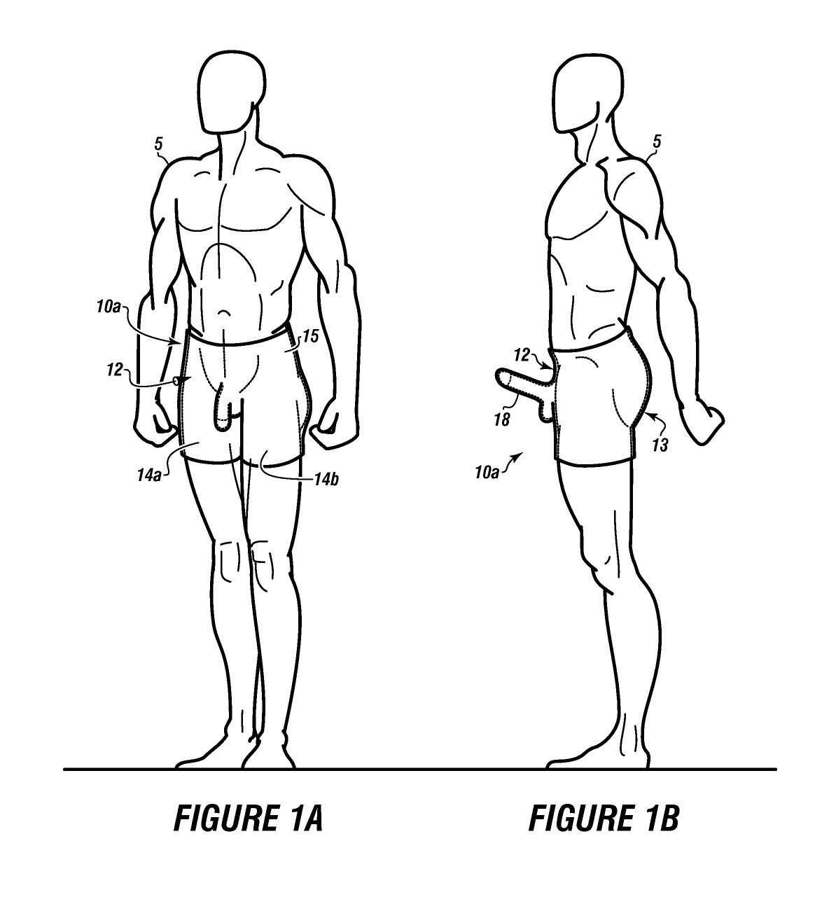

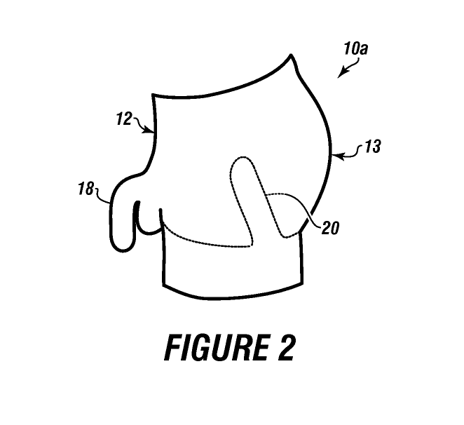

Turning now to the Figures, FIG. 1A depicts a front view of a flexible male undergarment on a male. FIG. 1B depicts a side view of view of the flexible male undergarment on a male. FIG. 2 depicts a side view of the flexible male undergarment.

Referring to FIGS. 1A, 1B, and 2, the flexible male undergarment 10a can include a front portion 12 and a back portion 13.

The front portion 12 and the back portion 13 can be connected to one another forming a first leg portion 14a, a second leg portion 14b, and a hip portion 15.

An integral front extension 18 can be located on the front portion 12. The integral front extension 18 can be configured to receive at least a penis of the wearer 5.

An integral back extension 20 can be located on the back portion 13. The integral back extension 20 can be configured to allow penetration of the wearer's anus and prevent direct contact with the skin of the anus. The integral back extension 20, when not used, can remain inserted in the undergarment so that it can be hidden and can lay flat against the skin and the flexible male undergarment.

The flexible male undergarment 10a can fit snugly against and partially cover a body of the wearer while simultaneously providing a tactile sensation to the wearer.

The flexible male undergarment 10a can be made from polymeric material.

The flexible male undergarment 10a can be worn prior to a date under an ordinary undergarment. The flexible male undergarment can be configured to provide full protection of the wearer from possible sexually transmitted diseases and other diseases that can result from contact with another person's skin, bodily fluids, or the like.

The flexible male undergarment 10a can have a moisturizing solution located thereon.

The moisturizing solution can be a powder like starch and calcium carbonate, or a moisturizing solution like Aloe Vera, Hydrogel, Lanolin oil and vitamin E or a solution having Aloe Vera, Lanolin oil, Vitamin E, or combinations thereof. The moisturizing solution can be applied to the formed flexible undergarment so that when the flexible male undergarment is worn the moisturizing solution is in contact with the wearer's skin.

An outer portion of the flexible male undergarment can have a flavor coating located thereon. The flavor coating can be sweet bubble gum, honey, or fruit products.

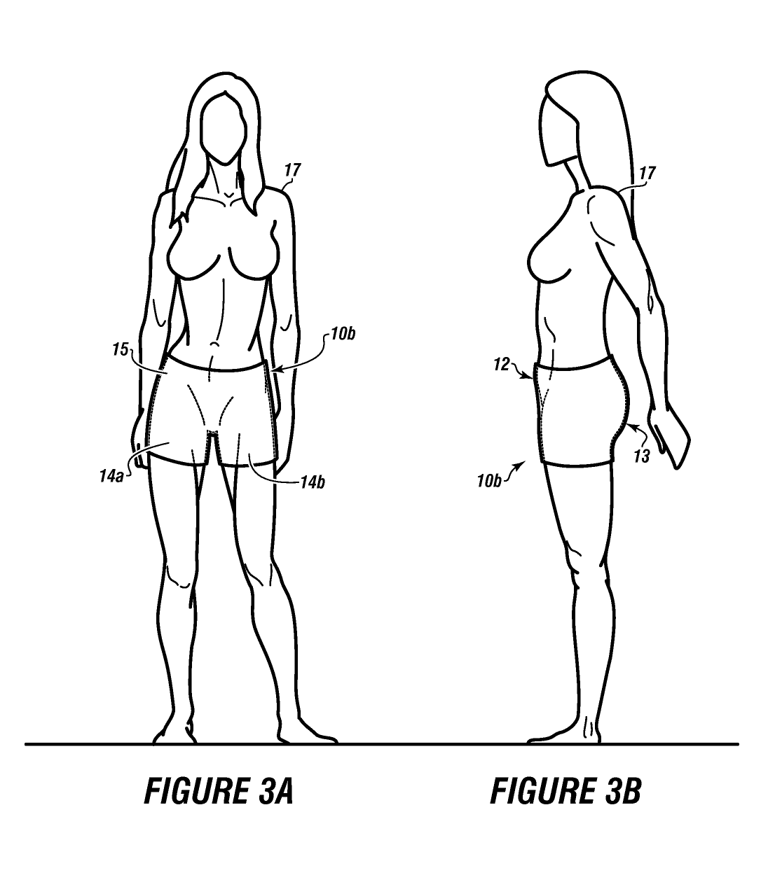

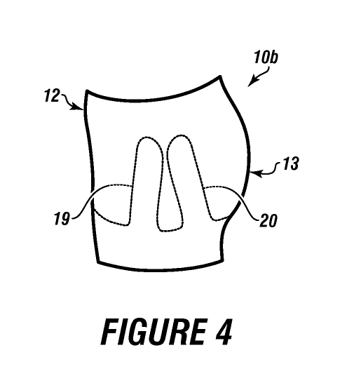

FIG. 3A depicts a front view of a flexible female undergarment on a female. FIG. 3B depicts a side view of the flexible female undergarment on a female. FIG. 4 is a side view of the flexible female undergarment.

Referring to FIGS. 3A, 3B, and 4, the flexible female undergarment 10b can include the front portion 12, the back portion 13, the first leg portion 14a, the second leg portion 14b, and the hip portion 15, and the integral back extension 20.

In addition, an integral middle extension 19 can be located on the front portion 12.

The integral middle extension 19 can be configured to allow vaginal penetration of a wearer 17 and prevent direct contact with the skin of the vagina. The integral middle extension 19 can remain flat between skin and the flexible female undergarment when not used.

The flexible female undergarment 10b can be worn prior to a date under an ordinary undergarment. The flexible female undergarment 10b can be configured to provide full protection of the wearer from possible sexually transmitted diseases and other diseases that can result from contact with another person's skin, bodily fluids, or the like.

The flexible female undergarment 10b can be configured to allow vaginal penetration, anal penetration or both while preventing skin to skin contact and transfer of bodily fluids.

The flexible female undergarment 10b can have a moisturizing solution located thereon. The moisturizing solution can be a powder like starch and calcium carbonate, or a moisturizing solution like Aloe Vera, Hydrogel, Lanolin oil and vitamin E or a solution having Aloe Vera, Lanolin oil, Vitamin E, or combinations thereof. The moisturizing solution can be applied to the formed flexible female undergarment so that when the flexible undergarment is worn the moisturizing solution is in contact with a wearer's skin.

An outer portion of the flexible female undergarment can have a flavor coating located thereon. The flavor coating can be sweet bubble gum, honey, or fruit products.

While these embodiments have been described with emphasis on the embodiments, it should be understood that within the scope of the appended claims, the embodiments might be practiced other than as specifically described herein.

What is claimed is:

1. A flexible male undergarment for sexual activity when penetration is intended comprising:
a. a front portion;
b. a back portion connected to the front portion, forming two leg portions and a hip portion configured to receive a lower portion of a body and to cover at least a portion thereof;
c. an integral front extension on the front portion, wherein the integral front extension is configured to receive at least a penis of a wearer;
d. an integral back extension on the back portion, wherein the integral back extension is configured to allow penetration of the wearer's anus and prevent direct contact with skin of the anus; and
e. wherein the flexible male undergarment fits snugly against and partially covers the body of the wearer while simultaneously providing a tactile sensation to the wearer, and wherein the flexible male undergarment is made from polymeric material.

2. The flexible male undergarment of claim 1, wherein the flexible male undergarment is configured to cover a portion of the wearer's stomach, an upper portion of the wearer's thighs, the wearer's genitals, and the wearer's anus.

3. The flexible male undergarment of claim 1, wherein the front portion and the back portion are seamless.

4. The flexible male undergarment of claim 1, wherein the flexible male undergarment has a thong shape, a brief shape, or a boxer shape.

5. The flexible male undergarment of claim 1, wherein the polymeric material is a natural rubber latex or a synthetic rubber latex or synthetic rubber.

6. The flexible male undergarment of claim 5, wherein the polymeric material has a thickness that is from 1/64 of an inch to ¼ of an inch.

7. The flexible male undergarment of claim 6, wherein the polymeric material is polypropylene, a polyamide, a polyester, homopolymers, copolymers, or combinations thereof.

8. The flexible male undergarment of claim 7, wherein the polymeric material is a film.

9. The flexible male undergarment of claim 5, wherein the synthetic rubber latex comprises a member of the group consisting of: acrylonitrile, polyurethane, polychloroprene, or polyvinyl chloride.

10. A flexible female undergarment for sexual activity when penetration is intended comprising:
a. a front portion;
b. a back portion connected to the front portion, forming two leg portions and a hip portion configured to receive a lower portion of a body and to cover at least a portion thereof;
c. an integral middle extension on the front portion, wherein the integral middle extension is configured to allow vaginal penetration of a wearer and prevent direct contact with skin of a vagina;
d. an integral back extension on the back portion, wherein the integral back extension is configured to allow penetration of the wearer's anus and prevent direct contact with the skin of the anus; and
e. wherein the flexible female undergarment fits snugly against and partially covers the body of the wearer while simultaneously providing a tactile sensation to the wearer, and wherein the flexible female undergarment is made from polymeric material.

11. The flexible female undergarment of claim 10, wherein the flexible female undergarment is configured to cover a portion of the wearer's stomach, an upper portion of the wearer's thighs, wearer's genitals, and the wearer's anus.

12. The flexible female undergarment of claim 10, wherein the flexible female undergarment is seamless.

13. The flexible female undergarment of claim 10, wherein the flexible female undergarment has a thong shape, a brief shape, or a boxer shape.

14. The flexible female undergarment of claim 10, wherein the polymeric material is a natural rubber latex or a synthetic rubber latex or synthetic rubber.

15. The flexible female undergarment of claim 14, wherein the synthetic rubber latex comprises a member of the group consisting of: acrylonitrile, polyurethane, polychloroprene, or polyvinyl chloride.

16. The flexible female undergarment of claim 14, wherein the polymeric material has a thickness from 1/64 of an inch to ¼ of an inch.

17. The flexible female undergarment of claim 16, wherein the polymeric material is polypropylene, a polyamide, a polyester, homopolymers, copolymers, or combinations thereof.

18. The flexible female undergarment of claim 17, wherein the polymeric material is a film.

19. A method of manufacturing a flexible undergarment with at least two extensions integrally formed therein for protecting a wearer from transmission of viral, fungal, and bacterial sexual diseases, comprising the steps of:
a. pouring polymeric material into a mold, wherein the mold is configured to form the flexible undergarment comprising:
(i) a front portion;
(ii) a back portion connected to the front portion, forming two leg portions and a hip portion configured to receive a lower portion of a body and to cover at least a portion thereof;
(iii) an integral back extension on the back portion, wherein the integral back extension is configured to allow penetration of the wearer's anus and prevent direct contact with skin of the anus; and
(iv) an integral middle extension on the front portion, wherein the integral middle extension is configured to allow vaginal penetration of the wearer and prevent direct contact with skin of a vagina, or an integral front extension on the front portion, wherein the integral front extension is configured to receive at least a penis of the wearer;
b. drying the polymeric material forming the flexible undergarment;
c. applying a solution comprising Aloe Vera to the flexible undergarment, wherein the Aloe Vera is applied to the flexible undergarment so that when the flexible undergarment is worn the Aloe Vera is in contact with the wearer's skin; and
d. drying the solution.
